(19) United States
(12) Patent Application Publication
(10) Pub. No.: US 2006/0127900 A1
(43) Pub. Date: Jun. 15, 2006

(54) Method for detecting increased susceptibility to tumors

(75) Inventors: Axel Meye, Dersden (DE); Helge Taubert, Grossorner (DE); Timo Hillebrand, Honow (DE); Peter Bendzko, Berlin (DE); Katharina Kruger, Berlin (DE); Matthias Kappler, Halle (DE); Manfred Wirth, Dresden (DE)

Correspondence Address: NORRIS, MCLAUGHLIN & MARCUS, P.A., 875 THIRD AVE, 18TH FLOOR, NEW YORK, NY, 10022, US

(73) Assignee: INVITEK GESELLSCHAFT FUR BIOTECHNIK & BIODESIGN MBH, Berlin (DE)

(21) Appl. No.: 10/518,317

(22) PCT Filed: Jun. 16, 2003
(86) PCT No.: PCT/EP03/06306

§ 371 Date: Jan. 3, 2006

(30) Foreign Application Priority Data
Jun. 18, 2002 (DE) 102 28 081.9

(51) Int. Cl. C12Q 1/68

(52) U.S. Cl. 435/6

(57) ABSTRACT

The invention relates to a method for detecting increased susceptibility to tumours by specifically detecting a polymorphism in the position 354 A ? G in the exon 12 of the human murine double minute-2 (MDM2) gene. Said polymorphism represents a hereditary marker for increased risk of cancer in humans. The invention also relates to the use of said tumour susceptibility marker for developing in vitro and in vivo test systems which integrate said markers, in a specific manner, into diagnostic, prognostic and possibly therapeutic methods.

The invention relates to a method for detecting increased susceptibility to tumours by specifically detecting a polymorphism at position 354 A→G in exon 12 of the human murine double minute-2 (MDM2) gene. Said polymorphism represents a hereditary marker for increased risk of cancer in humans. The invention is also directed to the use of said tumour susceptibility marker for developing in vitro and in vivo test systems which integrate said marker, in a specific manner, into diagnostic, prognostic and possibly therapeutic methods.

The MDM2 gene was first identified in the spontaneously transformed 3T3DM mouse cell line on double minute chromosomes. It is known that the gene product MDM2 can transform mouse fibroblasts and lead to uncontrolled and tumour-inducing growth. The human MDM2 gene is localised on the chromosomal segment 12q13-14 and gained in importance when it was shown that it represents an important antagonist for the p53 tumour suppressor gene. Mutual regulation takes place via a feed-back loop, i.e. the p53 protein activates the transcription of the MDM2 gene, and the MDM2 protein formed can in turn cause the degradation of the p53 protein. The MDM2 protein shows ubiquitin-ligase activity for p53 , the latter being labelled for proteosomal degradation. As a result, very fine regulation of the expression of p53 protein is achieved, which is essential above all in embryogenesis. Thus, MDM2 knock-out mice are mortal, but the mice survive if they do not additionally carry a functionally active p53 gene. It is also known that, in addition to interaction with the p53 tumour suppressor, MDM2 also affects another tumour suppressor metabolic pathway, i.e. that of Rb-E2F-p16INK4A/p19ARF. MDM2 can thus bind to the Rb protein and prevent the Rb-mediated G1 cell cycle arrest or interact directly with the transcription factors E2F1/DP and induce cell transition into the S phase. Because of the negative regulation of both tumour suppressor pathways, which are affected in about 80% of all tumours, and numerous findings that prove that the MDM2 protein is tumourigenic, the MDM2 gene is favoured as a target for gene therapy.

In conclusion, it can be stated that specific regions of MDM2 can interact with numerous proteins, such as p53 , CBP/p300, pRb, p73, E2F1, DP1, the ribosomal L5 ribonucleoprotein particle, p14ARF and RNA. The specific functions of MDM2 in tumour genesis, the cell cycle and apoptosis are discussed in excellent reviews (Freedman et al., 1999, Momand et al., 2000, Juven-Gershon and Oren, 1999).

The role of MDM2 has been investigated particularly on sarcomas, i.e. malignant tumours of mesenchymal origin. Amongst malignant tumours, sarcomas show the highest amplification rate—20-30%—for the MDM2 gene. Overexpression of MDM2 in transgenic mice results in sarcoma development (independently of p53 status) in 38% of cases. MDM2 overexpression in sarcoma patients correlates significantly with the poorer survival of patients concerned, as has been shown in a multivariate Cox regression analysis (Würl et al., 1997).

Overall, little is yet known about which normal or tumour-specific metabolic pathways are affected by the MDM2 mRNA or the MDM2 protein. One way of investigating the function of genes is to analyse the effect of genetic alterations. However, the MDM2 gene has so far been investigated for genetic alterations, i.e. mutations or polymorphisms, only to a very small extent. An extensive literature search has revealed a total of only four publications on this topic. These are a negative finding (no genetic alteration found) in human primary tumours (Silva et al., 2000), rarely occurring point and insertion mutations in the zinc finger region of MDM2 (Schlott et al., 1997), one case of polymorphism in the 5′ untranslated region (Heighway et al., 1994) and another case of polymorphism in exon 10 in the zinc finger region (Taubert et al., 2000). This polymorphism was ascertained exclusively for soft-tissue sarcomas (compared to the polymorphic allele frequency in healthy control subjects). The polymorphism was associated with a trend toward shorter survival (38 months as opposed to 57 months among patients without polymorphism).

The invention has been based on the object of identifying tumour-associated mutations or polymorphisms of the human MDM2 gene and ascertaining their correlations with disease predispositions. Starting from these correlations, the intention is to develop a method for the molecular-genetic diagnosis of these disease predispositions. The goal is to establish a model as a result of which a prophylactic or palliative therapy will be implementable that can be of a both surgical and medicinal nature.

The invention is based on the realisation that the polymorphism A→G (GAA→GAG) occurring in codon 354 in exon 12 of the MDM2 gene (nucleotide 1373 of the sequence NM_{—}002392) is not confined to soft-tissue sarcomas, but correlates with the predisposition to various malignant tumour types and is surprisingly of hereditary nature, i.e. it is already conserved in the germ line.

It has been found that this polymorphism has a tendency to correlate in certain solid tumours of epithelial origin (prostate carcinoma entity), but is not confined to these and also is of key importance for susceptibility to further solid and haematological tumours.

The invention is realised in accordance with the claims. The invention is therefore directed to a method for detecting tumour susceptibility, which method is characterised in that a nucleic acid of a test subject is isolated and the sequence of the human MDM2 gene is genotyped with the aid of base exchange A→G (GAA→GAG) at position 354 in exon 12, and a highly specific and very sensitive determination of the allelic status of this polymorphic gene locus (distinction between homo- and heterozygosity) is effected preferably in a high-throughput process.

Genotyping is effected by sequencing or other methods suitable for the detection of point mutations. These include PCR-supported genotyping methods, e.g. allele-specific PCR, other genotyping methods using oligonucleotides [examples are dot blotting or oligonucleotide ligation assays (OLA)], methods using restriction enzymes and single nucleotide polymorphism (SNP) analysis by means of matrix-assisted laser desorption/ionisation mass spectrometry (MALDI) as well as in principle any available method for variant detection, including chip technology in all its technological embodiments.

On the basis of the above, the inventive method is suitable for determining a broad spectrum of highly different predispositions. In one embodiment of the invention, the method is used for detecting homozygous or heterozygous polymorphism A→G at position 354 (exon 12) as a sufficient criterion for the genetic predisposition to a potential tumour susceptibility, particularly as a sufficient criterion for the genetic predisposition to the potential tumour risk for the affected patient and for his descendants.

In a preferred variant, the method, by detecting homozygous or heterozygous polymorphism, is to be used as a sufficient criterion for potential tumour susceptibility to solid epithelial tumours, e.g. prostate carcinoma (PCa), breast carcinoma, cervical carcinoma and/or ovarian carcinoma. The method is particularly suitable for detecting tumour susceptibility to PCa.

The diagnostician specialising in molecular biology thus has a universal hereditary tumour marker at his disposal.

In accordance with the invention, it is possible, depending on the homozygous or heterozygous detection of certain haplotypes, to make an informative statement on genetic predisposition. Genetic advice based on molecular genetics is thus made possible.

Furthermore, the identification of this polymorphism may serve as the diagnostic basis for preventive measures.

Detection is effected with the aid of isolated nucleic acids, and DNA or RNA can be used. Isolated RNA is transcribed into mRNA and cDNA using methods familiar to those skilled in the art. After this, the DNA is sequenced.

On the basis of this knowledge, by using this variable (mutated) nucleotide DNA sequence, new classes of therapeutic agents can be developed according to the invention which are directed at genes that affect the pathways of the MDM2 gene and attack the MDM2 gene (or genes associated with it) and, via regulation of transcription and translation and to influence their efficiency, act preferably by regulating expression.

Some of the pathways of the genes affecting the MDM2 gene are known. These include, for example:

p53 -p14

Rb-p16INK4A/p19ARF-E2F

mdr-1.

This leads preferably to the development of therapeutic agents that are directed at the human MDM2 gene and attack it at position 354 A→G in exon 12 in the MDM2 gene.

Moreover, this invention is also directed to in vitro and in vivo test systems. These test systems express the sequence of the human MDM2 gene mutated at position 354 A→G in exon 12 and can be used for investigating diseases involving the MDM2 gene and for developing and testing individually specific therapeutic agents in general.

Such test systems are well-known to those skilled in the art and can be cell lines, xenotransplants and other animal models.

Without intending to be limiting, the invention will be explained in greater detail below, using the example of the detection of tumour susceptibility to prostate carcinomas (PCa).

In the performance of analyses of selected and preoperatively obtained blood DNA samples from patients with urological tumour samples, patients with diagnosed primary PCa with corresponding family anamnesis (no evidence of cases of PCa in the family) and corresponding therapy (mainly localised PCa that was removed by radical prostatectomy with a curative treatment goal; more rarely, cases of hormone-refractory PCa treated by chemotherapy) were found to have a higher heterozygosity rate than the normal population of the Federal Republic of Germany for the MDM2 SNP A→G at position 354 (exon 12).

In successively extended analyses, polymorphism has so far been clearly detected in 31 of 229 investigated DNA samples (13.5%). From this number of cases, it can be clearly concluded that the MDM2 polymorphism rate is over double that of the normal population (assuming that the control subjects examined to determine the heterozygosity rate in the healthy and young normal population also includes male subjects with a non-predictively definable PCa risk). This result means that the heterozygous gene locus is a potential tumour susceptibility factor for patients with sporadic PCa.

It has also been of interest to observe that, in two DNA samples of patients with advanced PCa, homozygous allele results were obtained (both alleles corresponded to polymorphism A→G at position 354 (exon 12)).

It has been possible to solve existing problems when using HTS in molecular screening to detect the individually specific allelic status at the MDM2 gene locus to be investigated. The determination of the investigated MDM2 polymorphism has been carried out in parallel with high sensitivity and with exact typing of existing homo- or heterozygosity in patient DNA. The selected methodology is simple and quick to perform and is remarkable for its high degree of reproducibility and stability.

Over and above this, the method can be linked highly integratively to fully automatic DNA extraction from nucleated blood cells and also has potential for separate or, in combination with molecular sample preparation, full automation. The preferred detection method is based on a DNA ELISA and subsequent indirect enzymatic detection of the hybridisation result in a 96-well format. However, this preferred embodiment of a DNA ELISA should not limit further scope for the process for the molecular screening of the MDM2 locus to be investigated. In the preferred embodiment (DNA ELISA), previously isolated genomic DNA from the patient blood samples under investigation was used for generating double-stranded DNA fragments, which flank the MDM2 gene locus under investigation, by means of PCR technology. The primer pair for PCR use contained a primer that was biotinylated at its 5′ position. The generated PCR fragment is thus also biotinylated after amplification. The PCR fragment is subsequently transferred to the surface of a 96-well microtest plate coated with streptavidin and covalently bound to the plate surface by biotinylation. The double-stranded DNA fragment bound to the plate surface is denatured by addition of an NaOH solution and the unbound DNA single strand is removed in a brief washing step. The covalently bound DNA single strand serves thereafter as the target sequence for a base-complementary hybridisation reaction for the genotyping of the MDM2 status. Hybridisation is subsequently carried out in each case with two FITC-labelled oligonucleotide probes/amplified samples, which are base-complementary to both potentially possible MDM2 allele variants at the MDM2 locus under investigation. The hybridisation reaction is indirectly detected enzymatically by means of an enzyme-conjugated anti-FITC antibody reaction with subsequent substrate conversion. The existing allelic status is identified by evaluation of the colour changes or their intensities in the respective wells at the end of the reaction. On the basis of the colour pattern, it is possible to clearly identify the presence of homozygous or heterozygous bearers of characters. The method makes it possible to investigate 48 patient DNAs simultaneously with a single 96-well plate. The reproducibility and stability of the method has been demonstrated in the context of the large magnitudes of samples investigated, inclusive of series of blank tests. With the aid of additional DNA sequencing, the results generated in the DNA ELISA have been clearly verified for a series of samples.

The test result has been re-evaluated for all conspicuous samples and a representative number of inconspicuous samples and 100% confirmed independently using a different recognised detection system (direct PCR DNA sequencing, ALF Express, PharmaciaBiotech).

In addition, in further blank tests, multiple DNA aliquots of the same test subjects and negative controls have been determined in dependent and independent test series. These investigations also have yielded a complete match of qualitative results, which underlines the sensitivity of the selected detection method. Farther-reaching studies are currently underway, with which definitive statistical evaluations for over 400 patients with detectable sporadic PCa are being carried out.

Current tests on other carcinoma types have confirmed the association of the occurrence of this gene polymorphism with an elevated tumour rate for further solid epithelial tumour entities. For instance, in 5 of 32 blood DNA samples investigated so far (15.6%) of patients with breast, cervical or ovarian carcinoma, a heterozygous MDM2 allelic status has been demonstrated.

To demonstrate the specificity of the described PCR-ELISA analysis results, a subject subpopulation with known allelic status at the polymorphic MDM2 gene locus has been re-evaluated. In addition, attempts have been made by increasing the control group to define the precise heterozygosity frequency in the normal population (all the DNA samples described in this project came from healthy control subjects and tumour patients from the catchment area of Saxony and Saxony-Anhalt in 1996-2001 and were obtained with the patients' written consent and archived anonymously after DNA preparation). The DNA samples employed for this have come exclusively from blood donors with the associated strict inclusion criteria for the associated blood donations (no evidence of a tumour disease at the time of donation or before, no known family diseases, no elevated natural exposure to radiation and other mutagenic/carcinogenic substances at work or at home).

Of 108 blood DNA samples from normal subjects, a proportion of which had already been investigated independently for MDM2 polymorphism by Taubert et al. (2000) by means of sequencing and restriction digestion, all of the heterozygous DNA samples detected at the time have been clearly verified by PCR-ELISA. Furthermore, the polymorphism findings from 31 DNA samples of WTS tissue from this prior study have been confirmed with 100% specificity. In addition, from some of these WTS patients (n=6) whose tumour tissue DNA showed MDM2 polymorphism, corresponding DNA blood samples have been analysed and again have all been found to be positive. It can thus be assumed that the polymorphism detected in WTS patient tissue DNA samples is highly probably of hereditary nature, i.e. the polymorphism is conserved in the germ line.

Prostate carcinoma (PCa) is the second most frequently occurring cancer disease affecting men in Central Europe and, owing to its growing incidence in recent years, has been gaining in importance. In the USA, it is now the most frequently diagnosed tumour type and represents after lung carcinoma the tumour entity with the highest tumour-related fatality rate. In 80% of cases, the disease is diagnosed in men over 65 years of age. While the localised PCa is curable by removal of the prostate, curative treatment is rarely possible in cases of tumours that are locally advanced or accompanied by metastases. The 3-year survival rate for a tumour with metastases is only 40%. In cases of PCa, metastases spread via the blood stream and lymph tracts. The primary settlement locations for lymphogenic metastases are the pelvic lymph nodes. Haematogenic micrometastases mainly affect the skeletal system and above all the pelvis and spine as well as individual organs such as the liver and lungs. In the case of PCa with metastases, operative and medicinal therapies aim to suppress the formation and action of the hormone testosterone, which is the main cause of the proliferation of prostate and PCa cells. The correct determination of the tumour stage, i.e. whether it is a localised PCa or already developing metastases, is therefore absolutely essential for the subsequent form of treatment. The current examination methods used during the primary diagnosis of PCa are digital rectal examination, determination of the tumour marker PSA (prostate-specific antigen) in the serum and, in the case of removed biopsy material, its histopathological inspection and, in individual cases, diagnostic pelvic lymphadectomy and optional MRT and CT or bone scintigraphy. Until now, it has not been possible to diagnostically detect incipient metastasis formation (disseminated PCa cells in the blood, low invasion of the regional lymph nodes) in the blood or, in the case of the lymph nodes, detection is only possible postoperatively by histopathological investigation. The imaging techniques (CT, MRT) available for preoperative detection have a low sensitivity, which ranges from 22-26% and only permits the representation of extensive metastases. Since metastasis development is clearly dependent on the tumour stage, tumour volume and tumour grading, these—apart from histological differentiation (Gleason score)—are the most important factors referred to in determining the patient's prognosis.

In addition to digital rectal examination, the determination of the tumour marker PSA (prostate-specific antigen) is a highly selective and sensitive standard method for early diagnosis of PCa. However, the prostate-specific antigen is not a cancer-specific but a tissue-specific marker of the prostate. Elevated PSA serum values suggest the presence of PCa. Distinguishing between BPH and carcinoma with the aid of the PSA value is particularly difficult in the 2-10 ng/ml range, as BPH more frequently occurs with increasing age and the PSA value rises with increasing age due to natural prostate growth. The expression of PSA is regulated by the hormones testosterone and dihydrotestosterone (DHT). During the hormone treatment of patients (inhibition of the action of testosterone, dihydrotestosterone), the PSA value in the serum declines.

Owing to the health policy importance of PCa (particularly in the western industrialised nations), the lack of tumour-specific markers and the known tumour-biological and cellular heterogeneity of the tumour, there is an intensive search in the field of clinical research into PCa, which is focused among other things on identifying further genetic and epigenetic cofactors for sporadic and hereditary PCa. Particularly in the USA, there are well-characterised families with an enhanced PCa incidence, permitting far-reaching human genetic studies into (family-related) PCa.

The present invention discloses a universal hereditary tumour marker with polymorphism A→G (GAA→GAG) at position 354 in exon 12 of the MDM2 gene, particularly for PCa. It has been shown that polymorphism shows a strict association with PCa. By detecting this polymorphism, it is possible to make more reliable statements on the genetic predisposition to PCa and possible associated clinical pictures.

### REFERENCES

Freedman D. A., Wu L., Levine A. J. (1999), Functions of the MDM2 oncoprotein. Cell Mol. Life Sci. 55: 96-107.
Juven-Gershon T., Oren M. (1999), MDM2: The ups and downs. Mol. Med. 5: 71-83.
Momand J., Wu H. H., Dasgupta G. (2000), MDM2—master regulator of the p53 tumour suppressor protein. Gene 242: 15-29
Wörl P., Meye A., Berger D., Bache M., Lautenschläger C., Schmidt H. (1997), Prognostic relevance of C-terminal MDM2 detection is enhanced by positivity in soft tissue sarcomas. Diagn. Mol. Pathol. 6: 249-54.

1. A method for detecting tumour susceptibility, characterised in that a nucleic acid of a subject is isolated and the sequence of the human MDM2 gene is genotyped using the base exchange A→G (GAA→GAG) at position 354 in exon 12, and in which method a determination of the allelic status of this polymorphic gene locus is effected.

2. The method according to claim 1, characterised in that the detection of homozygous or heterozygous polymorphism (mutation) is used as a sufficient criterion for the genetic predisposition to a potential tumour susceptibility.

3. The method according to claim 1, characterised in that the detection of homozygous or heterozygous polymorphism (mutation) is used as a sufficient criterion for the genetic predisposition to a potential tumour risk for the subject and his descendants.

4. The method according to claim 1, characterised in that the detection of homozygous or heterozygous mutation is used as a sufficient criterion for a potential tumour susceptibility to prostate carcinoma, breast carcinoma, cervical carcinoma and/or ovarian carcinoma.

5. The method according to claim 1, characterised in that the genotyping is effected by sequencing the DNA or by other methods that are suitable for the detection of point mutations.

6. The method according to claim 1, characterised in that the genotyping is effected by DNA-ELISA using multiple, highly specific amplification primers and labelled hybridisation probes.

7. Therapeutic agents, which agents are directed at genes that affect the pathways in terms of the MDM2 gene and/or attack polymorphism A→G (GAA→GAG) at position 354 in exon 12 of the MDM2 gene or the genes associated therewith and, via regulation of transcription and translation and to influence their efficiency, act preferably by regulating expression.

8. Therapeutic agents, which agents are directed at the human MDM2 gene and attack the exchanged position A→G (GAA→GAG) at position 354 in exon 12 of the MDM2 gene and, via regulation of transcription and translation and to influence their efficiency, act preferably by regulating expression.

9. In vitro and in vivo test systems, which systems express the human MDM2 gene in the form having the mutation (polymorphism) A→G (GAA→GAG) at position 354 in exon 12 of the MDM2 gene, said test systems being used for investigating diseases involving the MDM2 gene and for developing and testing individually specific therapeutic agents in general.
